(19) United States
(12) Patent Application Publication
(10) Pub. No.: US 2022/0023107 A1
(43) Pub. Date: Jan. 27, 2022

(54) INTELLIGENT DIAPER MONITORING DEVICE

(71) Applicant: Chung-Ping Lai, Hsinchu County (TW)

(72) Inventors: Kuo-Hsin Chang, Chiayi County (TW); Chung-Ping Lai, Hsinchu County (TW)

(73) Assignee: Chung-Ping Lai

(21) Appl. No.: 17/034,020

(22) Filed: Sep. 28, 2020

(51) Int. Cl. A61F 13/42; G08B 21/20; G06K 19/07; G06K 19/077; H01Q 1/22

(52) U.S. Cl. CPC A61F 13/42; G08B 21/20; A61F 2013/424; G06K 19/07786; H01Q 1/2225; G06K 19/0724

(57) ABSTRACT

The invention provides a diaper monitoring device for detecting wetness of a diaper. The device includes an RFID tag, an RFID reading module, a first communication module, a power adapter, an application program, operating at a user end and a control server. The RFID reading module has a scan program. The scan program controls the RFID reading module to scan the RFID tag with the RF signal with various frequencies according to a scan parameter and sends a number of successful scans to the control server. The control server makes a notification indicating that the diaper is wet when the number of successful scans is less than a threshold. A user can adjust the scan parameter through the application program to modify sensitivity of diaper wetness degree.

### TECHNICAL FIELD

The invention relates to wetness detection of diapers, particularly to a device which can monitor wetness of a diaper and make a notification when the diaper is wet.

### RELATED ART

Conventional diapers can absorb water or urine but cannot actively notify the diaper being wet. Some diapers can show the diaper being wet by means of color changing after absorbing water or urine. Taiwan patent No. 1504386 provides a diaper with a detection function, which associates with an RFID reader. This diaper is installed with a sensing circuit connecting with a removable RFID tag. The sensing circuit will form a short circuit after the diaper absorbs urine. The RFID reader reads the RFID tag to detect variation of impedance between sensing wires and sends out a notification signal.

Taiwan patent No. 1533857 utilizes that the variation of the dielectric coefficient of the diaper waterproof layer will change the impedance of the metal antenna to further change the wavelength of the electromagnetic signals sent by the RFID tag when the absorbent has soaked. Although this means is different from TW 1504386, civil RFID tags use the UHF band, its wavelength is too short to pass through water molecules. Thus, water molecules will absorb the electromagnetic waves of this band to make the antenna fail to receive electromagnetic waves because the tag antenna and water form a big absorbent when the tag is attached on an object containing water. At this time, the transmitting distance of the electromagnetic waves will be very short. Water absorbs electromagnetic waves so as to shorten the transmitting distance of electromagnetic waves. This makes an RFID tag attached on a diaper cannot be read or activated. It is hard to judge if the interruption results from absorbed water or not.

U.S. Pat. No. 10,130,524 provides a diaper monitoring device with a shiftable-frequency RFID tag. The shiftable-frequency RFID tag has different reading frequency bands when a diaper is wet or not wet. The RFID tag cannot be read when the diaper is not wet. Conversely, the RFID tag can be read when the diaper is soaked by urine. Thus, the device can make a notification to show the diaper being wet.

Detection of a diaper being wet is used for being aware of replacing a diaper to a user, especially to a watcher of a newborn baby. In practice, a wetness degree depends on various personal judges. On the other hand, different wetness degrees make different people feel different. For example, for some people, a soaked diaper may be just a little wet and does not need to replace yet, but for other people, the same diaper is deemed very wet and needs to replace immediately.

Basically, the abovementioned prior arts utilize the principle of a wet diaper affecting the reading performance of an RFID tag. A diaper is judged wet and a notification is shown if the diaper is wet enough to affect the reading performance of an RFID tag. However, this principle does not consider different people may have different feelings and acceptances. Also, the known prior arts do not allow to adjust the sensitivity of the detecting device to satisfy actual requirements.

### SUMMARY OF THE INVENTION

An object of the invention is to provide a diaper monitoring device, which can monitor wetness of a diaper and make a notification when the diaper is wet enough to reach a predetermined threshold.

To accomplish the above object, the diaper monitoring device of the invention includes:

a radio-frequency identification (RFID) tag, attached on or implanted in the diaper;

an RFID reading module, used for sending a radio-frequency (RF) signal and receiving an echo signal from the RFID tag, having a scan program operated by the RFID reading module, and the scan program automatically controlling the RFID reading module to scan the RFID tag with the RF signal with various frequencies according to a scan parameter and recording a number of successful scans;

a first communication module, electrically connected to the RFID reading module;

a power adapter, electrically connected to the RFID reading module for converting an external power into a driving power to supply the RFID reading module;

an application program, operating at a user end; and

a control server, provided with a second communication module communicating and connecting with the first communication module, and bidirectionally communicating with the RFID reading module and the application program through the second communication module;

wherein the number of successful scans corresponding to the scan parameter is sent to the control server as a scan result, the RFID module bidirectionally communicates with the control server through the first communication module, the control server receives the scan result from the RFID reading module and sends both an operation command and the scan parameter from the application program to the RFID reading module, the control server sends out a first signal indicating that the diaper is wet when the number of successful scans of the scan result is less than a predetermined threshold of the scan parameter corresponding to the scan result, the application program is operated at a digital apparatus for obtaining the first signal and generates a notification corresponding to the first signal, the application program generates the operation command and sends both the operation command and the scan parameter to the control server.

Preferably, the scan parameter includes multiple RF powers, multiple transmitting frequencies corresponding to each RF power and the predetermined threshold corresponding to each RF power.

Preferably, the RFID tag is scanned with the RF signal of one of the RF powers of the scan parameter, and the RF signal comprises multiple RF signals of the various transmitting frequencies.

Preferably, the RFID tag is scanned with the RF signal of ones of the RF powers of the scan parameter, and the RF signal comprises multiple RF signals of the various transmitting frequencies.

Preferably, each of the first communication module and the second communication module is implemented by a Wi-Fi communication module, a 4G communication module or a Bluetooth® module.

Preferably, the application program is operated in a smartphone, a tablet or a wearable device.

Preferably, the RFIS tag is a UHF RFIS tag.

Preferably, the RFID reading module comprises a microprocessor, an antenna, an RF module and a memory, the RF module comprises a modulator, a demodulator and an adjustable power amplifier, the microprocessor is electrically connected to the RF module, the memory and the first communication module, the antenna is electrically connected to the RF module, the microprocessor generates a signal to the RFID tag and sends to the RF module, the microprocessor operates the scan program stored in the memory, the microprocessor controls the RF module to adjust the RF power and the transmitting frequency according to the scan parameter, the modulator of the RF module modulates the signal to the RFID tag into a carrier to be the RF signal, the RF signal is transmitted to the RFID tag with the RF power and the transmitting frequency via the antenna, the antenna receives the echo signal from the RFID tag 10, which means the successful scan, and the successful scan is counted to be the number of successful scans.

Preferably, the RFID reading module and the first communication module are integrated into one piece.

In the invention, sensitivity of wetness degree of a diaper can be changed by adjusting the scan parameter through the application program. This can make the wetness degree of a diaper meet a user's sensation.

### BRIEF DESCRIPTION OF THE DRAWINGS

FIG. 1 is a block diagram of the invention;

FIG. 2 is a schematic view of the RFID tag of the invention and a diaper;

FIG. 3 is a flowchart of the scanning process of the invention; and

FIG. 4 is a correspondence graph of RF power-transmitting frequency of successful scans of the RFID tag under different wetness degrees of a diaper.

### DETAILED DESCRIPTION OF THE INVENTION

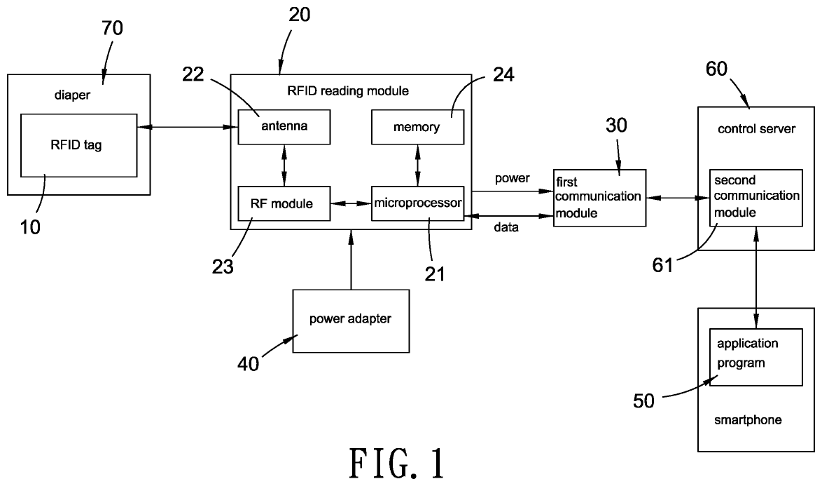

Please refer to FIG. 1. The diaper monitoring device of the invention includes an RFID (radio-frequency identification) tag 10, an RFID reading module 20, a first communication module 30, a power adapter 40, an application program 50 operating at a user end and a control server 60. The application program 50 may be a mobile application (also known as “app”) operating in a smartphone, a tablet or a wearable device.

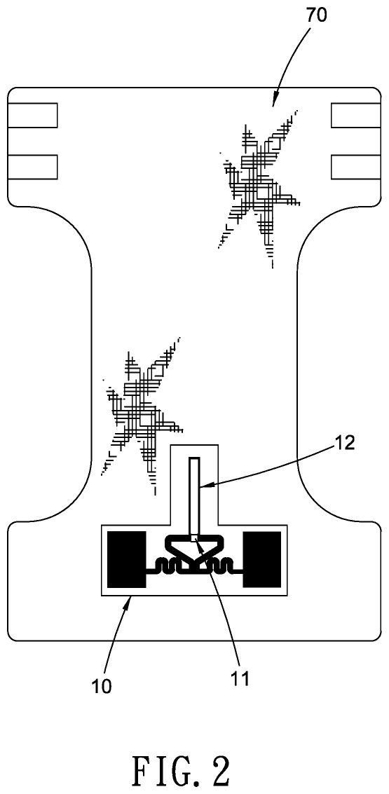

The RFID tag 10 includes an RFID chip 11 and an antenna 12 electrically connected to the RFID tag. The RFID tag 10 is attached on or implanted in a diaper 70. The embodiment shown in FIG. 2 is to attach the RFID tag 10 onto the diaper 70. When the RFID tag 10 is implanted in the diaper 70, the antenna 12 may be in contact with a urine absorbent, so the RFID chip 11 should be protected with a waterproof sealing material. Preferably, the RFID tag 10 is a UHF RFID tag.

The RFID reading module 20 is electrically connected to the first communication module 30. Preferably, the RFID reading module 20 and the first communication module 30 are integrated into one piece. The RFID reading module 20 bidirectionally communicates with the control server 60 through the first communication module 30. The RFID reading module 20 is used to send an RF signal to both scan the RFID tag 10 and receive an echo signal from the RFID tag 10. The RFID reading module 20 has a built-in scan program operated by the RFID reading module 20. The scan program automatically controls the RFID reading module 20 to scan the RFID tag with the RF signal with various frequencies according to a scan parameter, for example, automatically scan the RFID tag 10 at regular intervals, and then records the number of successful scans to the RFID tag 10. The number of successful scans corresponding to the scan parameter is sent to the control server 60 as a scan result. The term “successful scans” means that the RFID reading module 20 successfully receives the echo signal from the RFID tag 10, in substance, it means that the RFID reading module 20 successfully activates the RFID tag 10 and the RFID reading module 20 successfully reads the RFID tag 10. The scan parameter includes multiple RF powers, multiple transmitting frequencies corresponding to each RF power and a predetermined threshold corresponding to each RF power.

The RFID reading module 20 is substantially the same as an RFID reader in function and structure. As shown in FIG. 1, the RFID reading module 20 includes a microprocessor 21, an antenna 22, an RF (radio-frequency) module 23 and a memory 24. The RF module 23 includes a modulator, a demodulator and an adjustable power amplifier. Any person having ordinary skill in the art can realize and implement them. The microprocessor 21 is electrically connected to the RF module 23, the memory 24 and the first communication module 30. The antenna 22 is electrically connected to the RF module 23. The microprocessor 21 generates a signal to the RFID tag 10 and sends it to the RF module 23. The microprocessor 21 operates the scan program stored in the memory 24 and a preset scan parameter (the scan parameter can be reset through the application program by a user). The microprocessor 21 controls the RF module 23 to adjust its RF power and transmitting frequency according to the scan parameter. The modulator of the RF module 23 modulates the signal to the RFID tag 10 into a carrier to be an RF signal. The RF signal is transmitted to the RFID tag 10 with the abovementioned RF power and transmitting frequency via the antenna 22 to perform scan. After the RFID tag 10 is activated by the RF signal, the antenna 22 receives an echo signal from the RFID tag 10, which means a successful scan. Then the number of successful scans is counted. The echo signal received by the antenna 22 is sent to the demodulator, the demodulator demodulates the echo signal into a digital signal to send to the microprocessor 21. Data (usually including identification data of the RFID tag 10) obtained by the microprocessor 21 processing the digital signal is stored in the memory 24. The RFID reading module 20 further includes a system frequency generator which generates a working frequency of the system and a phase-locked loop (PLL) which generates a carrier of modulation.

The control server 60 serves as a background of the diaper monitoring device of the invention. The control server 60 may be a server, a personal computer or any other electronic devices which can operate a computer program. The control server 60 is provided with a second communication module 61 which communicating and connecting with the first communication module 30. In other words, the control server 60 can make bidirectional communication with the RFID reading module 20 and the electronic device operating the application program 50 via the second communication module 61. The control server 60 receives a scan result from the RFID reading module 20 and sends both an operation command (including a scan command implementing a manual scan) and the scan parameter from the application program 50 to the RFID reading module 20. The control server 60 sends out a first signal indicating that the diaper 70 is wet when the number of successful scans of the scan result is less than a predetermined threshold of the scan parameter corresponding to the scan result. The application program 50 generates a corresponding notification (such as numeral, text, optical or audio information) which is shown on an output device (such as display, speaker or indicator light) of a smartphone, a tablet or a wearable device to caution the user the diaper 70 being wet. On the other hand, a user may send a scan command to the control server 60 through the application 50 at any time, and then the control server 60 notifies the RFID reading module 20 to scan the diaper 70 immediately to implement a manual scan.

The power adapter 40 is electrically connected to the RFID reading module 20 for converting an external power into a driving power to supply the RFID reading module 20. The first communication module 30 and the second communication module 61 may be implemented by a Wi-Fi communication module, a 4G communication module or a Bluetooth® module to perform the bidirectional communication between the RFID reading module 20 and the control server 60 and the bidirectional communication between the control server 60 and the electronic device operating the application program 50.

The intelligent diaper monitoring device of the invention has features as follows:

1. As a preferred embodiment, the scan parameter stored in the RFID reading module 20 has at least one RF power. As another preferred embodiment, the scan parameter stored in the RFID reading module 20 has multiple RF powers. Each RF power in a preset frequency range such as 860M˜930 MHz (i.e., ultra-high frequency, UHF) at preset frequency intervals scans the RFID tag 10. In other words, transmitting an RF signal to scan the RFID tag 10 by with any RF power is equivalent to repeatedly scanning the RFID tag 10 with different transmitting frequency of RF signal at each scan.

2. Recording the scan result of the RF signal of each transmitting frequency of each RF power is equivalent to recording the number of successful scans of the RFID tag 10.

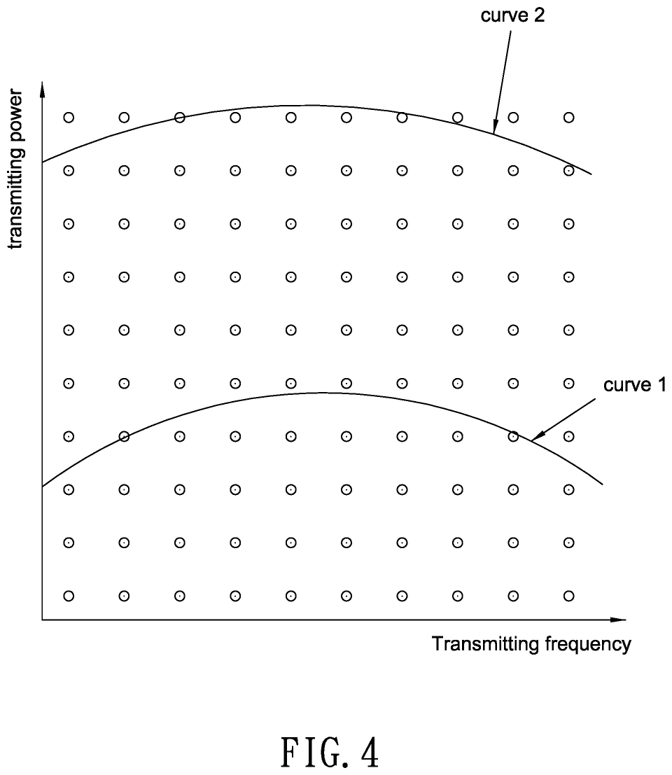

3. Whether a diaper 70 is wet or not is judged by monitoring the number of successful scans at each RF power. When a diaper 70 is dry, all the RF signal of each transmitting frequency of relatively lower RF powers can successfully activate the RFID tag 10 (i.e., the successful scans). When a diaper 70 is wet, the higher the wetness degree is, the higher the RF power needed to successfully activate the RFID tag 10 is. Please refer to FIG. 4, which is depicted by experimental data. As shown, a correspondence graph of RF power-transmitting frequency of successful scans of the RFID tag under different wetness degrees of a diaper can be seen. An RF signal sent with some transmitting frequency at some RF power is called a frequency point of a successful scan in the graph. Multiple frequency points are connected to be a curve shown in FIG. 4. In FIG. 4, the result of curve 1 stands for a diaper 70 being dry, and the result of curve 2 stands for a diaper 70 being wet.

A curve of activating power (the relationship between the RF power and the transmitting frequency of the activated RFID tag 10) of the RFID tag 10 on surfaces of different diapers 70 has certain randomness. Consistency is hard to appear. It is necessary to collect scan results of the RFID tag 10 in a wide frequency range to obtain an overall trend of variation of the activating power. On the other hand, when a diaper 70 which is attached by the RFID tag 10 is wet, its dielectric constant will become larger. This will make the RFID tag 10 unmatched (the reading performance varies) to increase the activating power.

Preferably, the invention sets different predetermined thresholds in the scan parameter. Each predetermined threshold has an exclusive power sector. According to magnitude of the RF power, the power sectors are divided into a low power sector, a middle power sector and a high power sector. Each power sector further includes multiple different transmitting powers. For example, RF powers of the low power sector is set to be 18 dBm, 21 dBm and 30 dBm, RF powers of the middle power sector is set to be 21 dBm, 24 dBm and 30 dBm, and RF powers of the high power sector is set to be 24 dBm, 27 dBm and 30 dBm. A user can select one of the power sectors according to actual conditions through the application program 50.

Another factor determining the RF power and its corresponding predetermined threshold is the reading distance of the RFID tag 10. When the middle power sector is selected, the scanning frequency range is 840-960 MHz, the frequency interval is 10 MHz, and the number of the RF signals at each RF power is 13. The predetermined thresholds corresponding to each RF power may be set as follows: when the RF power is 21 dBm, scan the RFID tag 10 with multiple (such as 13) different transmitting frequencies, if the number of successful scans is below 2; when the RF power is 24 dBm, the number of successful scans is below 4; or when the RF power is 30 dBm, the number of successful scans is below 6; the diaper 70 can be judged wet. Preferably, sensitivity of wetness degree of a diaper 70 can be changed by adjusting the scan parameter through the application program. This can make the wetness degree of a diaper 70 meet a user's sensation.

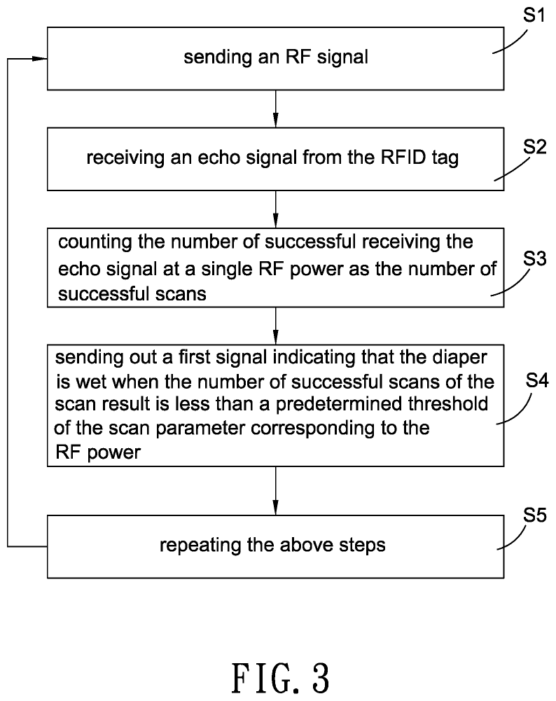

Steps of the scan program operated by the RFID reading module 20 is shown in FIG. 3 and listed as below:

S1) sending out an RF signal with a RF power predetermined by a scan parameter at multiple different transmitting frequencies;

S2) receiving an echo signal from the RFID tag 10 after sending out the RF signal with each transmitting frequency;

S3) counting the number of successful receiving the echo signal at a single RF power as the number of successful scans;

S4) sending out a first signal indicating that the diaper 70 is wet when the number of successful scans of the scan result is less than a predetermined threshold of the scan parameter corresponding to the RF power, otherwise sending out a second signal indicating that the diaper 70 is not wet; and

S5) repeating the above steps to implement periodically automatically scanning the diaper 70.

What is claimed is:

1. A diaper monitoring device for detecting wetness of a diaper, comprising:
a radio-frequency identification (RFID) tag, attached on or implanted in the diaper;
an RFID reading module, used for sending a radio-frequency (RF) signal and receiving an echo signal from the RFID tag, having a scan program operated by the RFID reading module, and the scan program automatically controlling the RFID reading module to scan the RFID tag with the RF signal with various frequencies according to a scan parameter and recording a number of successful scans;
a first communication module, electrically connected to the RFID reading module;
a power adapter, electrically connected to the RFID reading module for converting an external power into a driving power to supply the RFID reading module;
an application program, operating at a user end; and
a control server, provided with a second communication module communicating and connecting with the first communication module, and bidirectionally communicating with the RFID reading module and the application program through the second communication module;
wherein the number of successful scans corresponding to the scan parameter is sent to the control server as a scan result, the RFID module bidirectionally communicates with the control server through the first communication module, the control server receives the scan result from the RFID reading module and sends both an operation command and the scan parameter from the application program to the RFID reading module, the control server sends out a first signal indicating that the diaper is wet when the number of successful scans of the scan result is less than a predetermined threshold of the scan parameter corresponding to the scan result, the application program is operated at a digital apparatus for obtaining the first signal and generates a notification corresponding to the first signal, the application program generates the operation command and sends both the operation command and the scan parameter to the control server.

2. The diaper monitoring device of claim 1, wherein the scan parameter includes multiple RF powers, multiple transmitting frequencies corresponding to each RF power and the predetermined threshold corresponding to each RF power.

3. The diaper monitoring device of claim 2, wherein the RFID tag is scanned with the RF signal of one of the RF powers of the scan parameter, and the RF signal comprises multiple RF signals of the various transmitting frequencies.

4. The diaper monitoring device of claim 2, wherein the RFID tag is scanned with the RF signal of ones of the RF powers of the scan parameter, and the RF signal comprises multiple RF signals of the various transmitting frequencies.

5. The diaper monitoring device of claim 1, wherein each of the first communication module and the second communication module is implemented by a Wi-Fi communication module, a 4G communication module or a Bluetooth® module.

6. The diaper monitoring device of claim 1, wherein the application program is operated in a smartphone, a tablet or a wearable device.

7. The diaper monitoring device of claim 1, wherein the RFIS tag is a UHF RFIS tag.

8. The diaper monitoring device of claim 2, wherein the RFID reading module comprises a microprocessor, an antenna, an RF module and a memory, the RF module comprises a modulator, a demodulator and an adjustable power amplifier, the microprocessor is electrically connected to the RF module, the memory and the first communication module, the antenna is electrically connected to the RF module, the microprocessor generates a signal to the RFID tag and sends to the RF module, the microprocessor operates the scan program stored in the memory, the microprocessor controls the RF module to adjust the RF power and the transmitting frequency according to the scan parameter, the modulator of the RF module modulates the signal to the RFID tag into a carrier to be the RF signal, the RF signal is transmitted to the RFID tag with the RF power and the transmitting frequency via the antenna, the antenna receives the echo signal from the RFID tag 10, which means the successful scan, and the successful scan is counted to be the number of successful scans.

9. The diaper monitoring device of claim 1, wherein the RFID reading module and the first communication module are integrated into one piece.
